(19) United States
(12) Patent Application Publication
(10) Pub. No.: US 2026/0368796 A1
(43) Pub. Date: Nov. 19, 2026

(54) METHOD AND SYSTEM FOR RECOMMENDING SLEEP PRODUCT STATE PARAMETERS BASED ON LYING TEST

(71) Applicant: Shenzhen Stareep Smart Sleep Tech Co., Ltd., Shenzhen (CN)

(72) Inventors: Shijing Wang, Shenzhen (CN); Yanming Cai, Shenzhen (CN); Muyang Zhang, Shenzhen (CN); Xueyang Wang, Shenzhen (CN)

(21) Appl. No.: 19/409,864

(22) Filed: Dec. 5, 2025

(30) Foreign Application Priority Data
May 19, 2025 (CN) 202510644390.4

(51) Int. Cl. G06Q 30/0601; A47C 20/04; A47C 31/12; G01L 5/16

(52) U.S. Cl. CPC G06Q 30/0631; A47C 20/04; A47C 31/123; G01L 5/16

(57) ABSTRACT

The present disclosure relates to method and system for recommending sleep product state parameters based on lying test. The method comprises: in response to a user entering the lying cabin, performing a plurality of rounds of pressure detection on the user, and in a current round of the plurality of rounds of pressure detection, determining a sleep detection posture corresponding to the current round; guiding the user to lie on the adjustable mattress in the sleep detection posture, and performing a step-by-step adjustment on state parameters of the adjustable mattress to obtain pressure data collected by the pressure sensor array; collecting the user's physiological health data based on the heart rate and respiration belt; and determining recommended sleep product state parameters according to pressure data collected in the plurality of rounds of pressure detection and the physiological health data. The recommendation accuracy can be improved by adopting the method.

### CROSS-REFERENCE TO RELATED APPLICATION

The present disclosure claims priority to Chinese Patent Application No. 202510644390.4 filed with the China National Intellectual Property Administration on May 19, 2025, and entitled “METHOD AND SYSTEM FOR RECOMMENDING SLEEP PRODUCT STATE PARAMETERS BASED ON LYING TEST”, the contents of which are incorporated herein by reference in entirety.

### TECHNICAL FIELD

The present disclosure relates to the technical field of sleep product state parameter recommendation, and in particular, to a method and system for recommending sleep product state parameters based on lying test.

### BACKGROUND ART

One-third of a person's life is spent sleeping, and the quality of sleep is closely related to a person's mental and physical health. Sleep quality is not only affected by a person's own psychological and physiological states, but also closely related to sleep products. Therefore, it is particularly important to reasonably set state parameters of sleep products.

In traditional technologies, recommended state parameters are generally determined based on a user's human body characteristics (e.g., height, weight) in a static state. For example, a state parameter of a sleep product may be a firmness of the mattress, and a recommended firmness of the mattress is determined based on the height and weight.

However, there is a problem of inaccuracy when determining a recommended state parameter of a sleep product based on human body characteristics in a static state.

### SUMMARY

Based on this, for addressing the above technical problems, it is necessary to provide a method and device for recommending sleep product state parameters based on lying test, and a computer device, a computer-readable storage medium, and a computer program product, which may improve accuracy.

The present disclosure provides a method for recommending sleep product state parameters based on lying test, which is applied to a lying cabin. The lying cabin comprises an adjustable mattress, and a pressure sensor array and a heart rate and respiration belt are provided on the adjustable mattress. The method comprises:
in response to a user entering the lying cabin, performing a plurality of rounds of pressure detection on the user, and in a current round of the plurality of rounds of pressure detection, determining a sleep detection posture corresponding to the current round;
guiding the user to lie on the adjustable mattress in the sleep detection posture, and performing a step-by-step adjustment on state parameters of the adjustable mattress to obtain pressure data collected by the pressure sensor array;
collecting the user's physiological health data based on the heart rate and respiration belt; and
determining recommended sleep product state parameters according to pressure data collected in the plurality of rounds of pressure detection and the physiological health data.

The present disclosure provides a system for recommending sleep product state parameters based on lying test. The system comprises a lying cabin, an electric bed frame, an adjustable mattress, and a display screen.

The electric bed frame and the display screen are arranged in the lying cabin, and the adjustable mattress is placed on the electric bed frame. The adjustable mattress is provided with a pressure sensor array and a heart rate and respiration belt, and the adjustable mattress comprises a plurality of zones. Each zone corresponds to a different part of a human body, and each zone is provided with an airbag.

The electric bed frame is configured to independently lift a head and legs and adjust a tilt angle under a drive of servo motors for the head and legs.

The adjustable mattress is configured to perform at least one of a firmness adjustment and a height adjustment based on airbags arranged in the zones during a plurality of rounds of pressure detection on a user.

The pressure sensor array is configured to collect pressure data during each round of pressure detection.

The heart rate and respiration belt is configured to collect the user's physiological health data.

The display screen is configured to display sleep posture pressure distribution maps generated based on the pressure data and recommended sleep product state parameters generated based on the pressure data and the physiological health data.

The present disclosure further provides a device for recommending sleep product state parameters based on lying test. The device comprises a pressure detection module, a physiological health data collection module, and a state parameter output module.

The pressure detection module is configured to: when the user enters the lying cabin, perform the plurality of rounds of pressure detection on the user, and in the current round of the plurality of rounds of pressure detection, determine the sleep detection posture corresponding to the current round; and guide the user to lie on the adjustable mattress in the sleep detection posture, and perform the step-by-step adjustment on the state parameters of the adjustable mattress to obtain the pressure data collected by the pressure sensor array.

The physiological health data collection module is configured to collect the user’s physiological health data based on the heart rate and respiration belt.

The state parameter output module is configured to determine the recommended sleep product state parameters according to the pressure data collected in the plurality of rounds of pressure detection and the physiological health data.

The present disclosure further provides a computer device. The computer device comprises a memory and a processor. The memory stores a computer program, and when the processor executes the computer program, the steps of any method for recommending sleep product state parameters based on lying test provided in the embodiments of the present disclosure are implemented.

The present disclosure further provides a computer-readable storage medium. A computer program is stored on the computer-readable storage medium, and when the computer program is executed by a processor, the steps of any method for recommending sleep product state parameters based on lying test provided in the embodiments of the present disclosure are implemented.

The present disclosure further provides a computer program product. The computer program product comprises a computer program, and when the computer program is executed by a processor, the steps of any method for recommending sleep product state parameters based on lying test provided in the embodiments of the present disclosure are implemented.

For the above method, system, and device for recommending sleep product state parameters based on lying test, and the computer equipment, the storage medium, and the computer program product, when the user enters the lying cabin, by guiding the user to sleep in different sleep detection postures, the different rounds of pressure detection may be performed under different sleep detection postures, thereby obtaining the pressure data corresponding to the respective sleep detection postures. By obtaining the pressure data, the recommended sleep product state parameters may be obtained by combining the collected physiological health data. Since the recommended sleep product state parameters are determined by combining both the physiological health data and the pressure data, the recommended sleep product state parameters are accurate. Moreover, by taking different sleep postures into account, the sleep product state parameters recommended to the user, which are obtained from the pressure data corresponding to the respective sleep detection postures, are more accurate.

### BRIEF DESCRIPTION OF DRAWINGS

FIG. 1 is an application environment diagram of a method for recommending sleep product state parameters based on lying test in an embodiment;

FIG. 2 is a schematic flow diagram of a method for recommending sleep product state parameters based on lying test in an embodiment;

FIG. 3 is a schematic diagram of a system for recommending sleep product state parameters based on lying test in an embodiment;

FIG. 4 is an overall schematic flow diagram of a method for recommending sleep product state parameters based on lying test in an embodiment;

FIG. 5 is a structural block diagram of a device for recommending sleep product state parameters based on lying test in an embodiment; and

FIG. 6 is an internal structure diagram of a computer device in an embodiment.

### DETAILED DESCRIPTION OF EMBODIMENTS

To make the objectives, technical solutions, and advantages of the present disclosure clearer, the present disclosure will be further described in detail below with reference to the drawings and embodiments. It should be understood that the specific embodiments described herein are only used to explain the present disclosure and are not intended to limit the present disclosure.

A method for recommending sleep product state parameters based on lying test provided in the embodiments of the present disclosure may be applied to an application environment shown in FIG. 1. A lying cabin 102 communicates with a server 104 through a network. A data storage system may store data that the server 104 needs to process. The data storage system may be integrated into the server 104, or placed on a cloud or other servers. After a user enters the lying cabin 102, the lying cabin 102 may guide the user to lie on an adjustable mattress in a preset sleep detection posture, and then a pressure sensor array may collect pressure data. The lying cabin 102 may guide the user to switch between a plurality of sleep detection postures, so that the pressure sensor collects corresponding pressure data under each sleep detection posture. After the pressure detection is completed, the lying cabin 102 may guide the user to lie still, and a heart rate and respiration belt may collect the user's physiological health data in a static lying state. Then, the lying cabin 102 may send the pressure data and the physiological health data to the server 104. The server 104 may determine recommended sleep product state parameters based on the received pressure data and physiological health data, and return the recommended sleep product state parameters to the lying cabin. The lying cabin 102 may display the recommended sleep product state parameters. An employee holding a tablet may stand at an entrance of the lying cabin 102, and the user may scan a QR code on the tablet with a mobile phone to input information. The lying cabin 102 has an electric curtain, an electric bed frame, and a display screen, and the display screen may show information such as a pressure cloud map. The server 104 may be implemented by an independent server or a server cluster composed of a plurality of servers. The server 104 may also be a cloud server.

The method for recommending sleep product state parameters based on lying test may be applied to a system for recommending sleep product state parameters based on lying test. The system for recommending sleep product state parameters based on lying test includes a lying cabin, an electric bed frame, an adjustable mattress, and a display screen. To facilitate a better understanding of the present disclosure, the components of the system are introduced below. The lying cabin, also known as an intelligent simulation cabin, has a closed design and integrates an annular sound-insulating panel, an openable and closable electric curtain, a surrounding light-emitting diode (LED) guide light, and a speaker capable of three-dimensional voice prompts. The electric bed frame is disposed in the lying cabin. A head and legs of the electric bed frame are respectively driven by servo motors, for supporting independent lifting and tilt angle adjustment of the head and legs. The adjustable mattress is placed on the electric bed frame. The adjustable mattress is divided into a plurality of zones, with each zone corresponding to a part of a human body. Moreover, each zone is provided with an independently controlled airbag, for supporting dynamic adjustment of firmness and local height. A flexible pressure sensor array and a heart rate and respiration belt are provided on the adjustable mattress. The flexible pressure sensor array is used to collect pressure data, and the heart rate and respiration belt is used to collect physiological health data. The display screen is also disposed in the lying cabin, and is used to display a sleep posture pressure distribution map generated based on the pressure data and recommended sleep product state parameters generated based on the pressure data and the physiological health data.

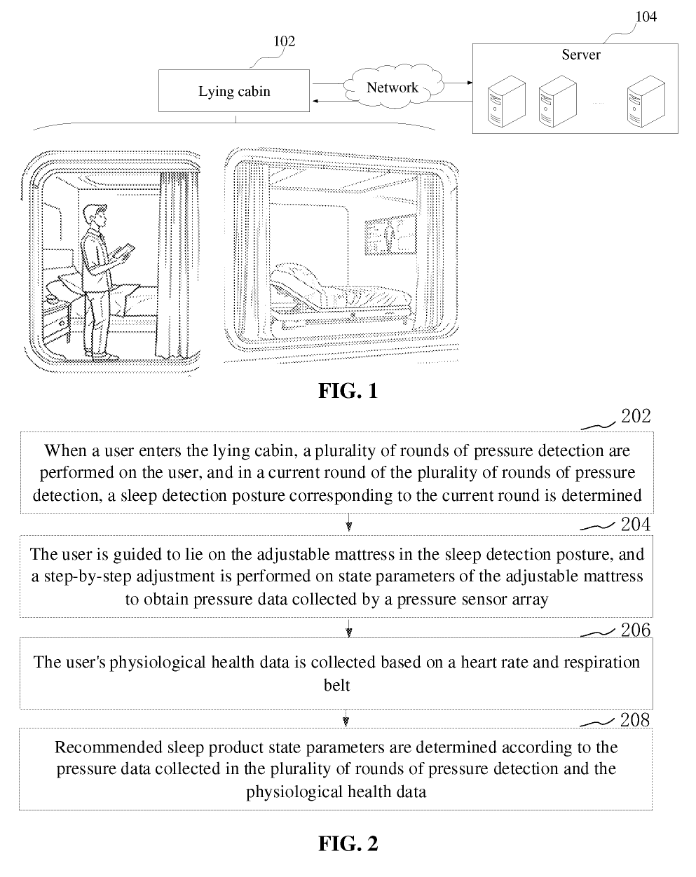

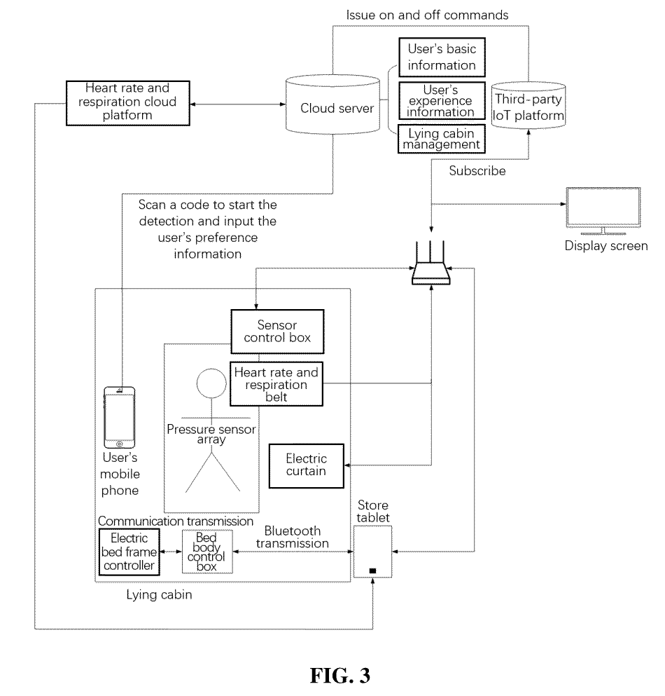

In one of the embodiments, as shown in FIG. 2, a method for recommending sleep product state parameters based on lying test is provided. Taking the application of the method to the lying cabin in FIG. 1 as an example, the method includes the following steps.

In Step 202, when a user enters the lying cabin, a plurality of rounds of pressure detection are performed on the user, and in a current round of the plurality of rounds of pressure detection, a sleep detection posture corresponding to the current round is determined.

The sleep detection posture refers to a sleeping posture. For example, the sleep detection posture may specifically be lying on one's back, lying on one's side, lying prone, semi-side-lying, etc.

Specifically, when the user enters the lying cabin, the plurality of rounds of pressure detection may be performed on the user. For example, a first round of pressure detection may be performed on the user. After the first round of pressure detection is completed, a second round of pressure detection is performed, and this process is repeated until a last round of pressure detection is completed. Different rounds of pressure detection correspond to different sleep detection postures. For example, the first round of pressure detection may correspond to lying on one's back, the second round of pressure detection may correspond to lying on one's side, and so on. When performing the pressure detection of the current round, the sleep detection posture corresponding to the current round may be determined.

In one of the embodiments, a sleep detection posture to which each round of pressure detection corresponds may be preset to obtain a correspondence between a round and a sleep pressure detection posture. Thus, when performing the pressure detection of the current round, the sleep detection posture corresponding to the current round may be determined based on the correspondence.

In Step 204, the user is guided to lie on the adjustable mattress in the sleep detection posture, and a step-by-step adjustment is performed on state parameters of the adjustable mattress to obtain pressure data collected by a pressure sensor array.

The step-by-step adjustment refers to a method of adjusting a certain parameter or state in a discrete and gradual manner, which has the characteristics of discreteness, accuracy, and repeatability. Discreteness means that the adjustment is not continuous and smooth, but changes according to a certain step size. For example, an amplitude of each adjustment is a fixed value or ratio. Accuracy means that a precise adjustment is achieved by controlling a magnitude of a step size. Repeatability means that the adjustment according to a set step size has good repeatability.

The state parameters refer to parameters for representing states of sleep products. The sleep products may include a mattress and a pillow. The states of the sleep products may be a firmness state of the mattress and a height state of the pillow. Therefore, the state parameters may specifically be a firmness of the mattress and a height of the pillow.

Specifically, in the pressure detection of the current round during the plurality of rounds of pressure detection, after the sleep detection posture corresponding to the current round is determined, the user may be guided to lie on the adjustable mattress in this sleep detection posture. For example, when the pressure detection of the current round corresponds to lying on one's back, an example of lying on one's back may be displayed on the display screen to guide the user to lie on their back. Further, after confirming that the user lies on the adjustable mattress in the sleep detection posture corresponding to the current round, the step-by-step adjustment may be performed on the state parameter(s) of the adjustable mattress, and after each adjustment, the pressure sensor array may collect pressures to obtain corresponding pressure data. For example, at least one of a firmness of the adjustable mattress and a height of a head airbag in the adjustable mattress may be adjusted, and after adjustment, the pressure sensor collects pressure data.

In Step 206, the user's physiological health data is collected based on a heart rate and respiration belt.

Specifically, the heart rate and respiration belt is disposed on the adjustable mattress. After the plurality of rounds of pressure detection are completed, the psychological health data of the user may be collected through the heart rate and respiration belt. For example, data such as the user's heart rate and respiratory rate is collected through the heart rate and respiration belt. The heart rate and respiration belt may be a flexible sensor, which is arranged at a position of the adjustable mattress corresponding to the user's heart.

In Step 208, recommended sleep product state parameters are determined according to the pressure data collected in the plurality of rounds of pressure detection and the physiological health data.

Specifically, after obtaining the pressure data and the physiological health data, the recommended sleep product state parameters, e.g., a recommended mattress firmness and a recommended pillow height may be determined based on the pressure data and the physiological health data.

In one of the embodiments, the user may also input preference information. The preference information includes preferred state data such as preferred mattress firmness and preferred pillow height as well as preferred sleep posture. The recommended sleep product state parameters are obtained by synthesizing the preference information, the pressure data, and the physiological health data.

In one of the embodiments, since the state parameters of the adjustable mattress are adjusted in a step-by-step manner under different sleep detection postures, pressure data corresponding to the respective state parameters is obtained for the same sleep detection posture. Furthermore, as pressure data may reflect a contact condition between a human body and a mattress as well as a degree of pressure concentration, for the same sleep detection posture, optimal pressure data in various pressure data under this sleep detection posture may be determined, and then optimal state parameters corresponding to the optimal pressure data are obtained. The optimal state parameters serve as optimal state parameters corresponding to the sleep detection posture. For example, in a case of lying on one's back, optimal pressure data may be screened out from various pressure data collected when the user lies on their back. When the optimal pressure data is collected, the state parameters of the adjustable mattress are determined and used as optimal state parameters corresponding to lying on one's back.

Further, after obtaining the respective optimal state parameters corresponding to various sleep detection postures, the respective optimal state parameters corresponding to various sleep detection postures may be synthesized to determine optimal state parameters corresponding to the pressure detection. For example, the respective optimal state parameters may be averaged to obtain the optimal state parameters corresponding to the pressure detection. Further, a preset correspondence between the physiological health data and the optimal state parameters may be obtained. When the physiological health data collected by the heart rate and respiration belt is obtained, the optimal state parameters corresponding to the physiological health data collected by the heart rate and respiration belt may be determined based on the preset correspondence. The recommended sleep product state parameters are determined based on the optimal state parameters corresponding to the physiological health data collected by the heart rate and respiration belt and the optimal state parameters corresponding to the pressure detection. For example, the optimal state parameters corresponding to the physiological health data collected by the heart rate and respiration belt and the optimal state parameters corresponding to the pressure detection are averaged to obtain the recommended sleep product state parameters.

In the above method for recommending sleep product state parameters based on lying test, when the user enters the lying cabin, by guiding the user to sleep in different sleep detection postures, the different rounds of pressure detection may be performed under different sleep detection postures, thereby obtaining the pressure data corresponding to the respective sleep detection postures. By obtaining the pressure data, the recommended sleep product state parameters may be obtained by combining the collected physiological health data. Since the recommended sleep product state parameters are determined by combining both the physiological health data and the pressure data, the recommended sleep product state parameters are accurate. Moreover, by taking different sleep postures into account, the sleep product state parameters recommended to the user, which are obtained from the pressure data corresponding to the respective sleep detection postures, are more accurate.

In one of the embodiments, performing the step-by-step adjustment on the state parameters of the adjustable mattress to obtain the pressure data collected by the pressure sensor array includes: performing a plurality of firmness adjustments on the firmness of the adjustable mattress to obtain pressure data collected by the pressure sensor array after each firmness adjustment; and performing a plurality of head height adjustments on the adjustable mattress to obtain pressure data collected by the pressure sensor array after each head height adjustment.

Specifically, during the pressure detection of the current round, a plurality of adjustments may be performed on the firmness of the adjustable mattress and the height of the head airbag in the adjustable mattress. For example, the firmness of the adjustable mattress may be adjusted in a 7-level firmness step manner, and the pressure sensor array collects corresponding pressure data after each firmness step. Subsequently, the plurality of head height adjustments may be performed on the adjustable mattress. For example, the height of the head airbag in the adjustable mattress may be adjusted in a 7-level height step manner, and the pressure sensor array collects corresponding pressure data after each height adjustment.

In one of the embodiments, a plurality of levels of firmness and a plurality of levels of height may be preset. For example, 7 levels of firmness and 7 levels of height may be preset. Among the 7 levels of height, a subsequent level of height is 1 centimeter higher than a previous level of height, with a total height range of the 7 levels being 0 to 6 centimeters. During the plurality of firmness adjustments, each level of firmness is maintained for a certain period, e.g., 2.5 seconds, so as to enable the pressure sensor array to fully collect pressure data.

In the above embodiments, during each round of pressure detection, by performing the plurality of adjustments on the firmness of the adjustable mattress and performing the plurality of adjustments on the head height, pressure data is collected after each adjustment, thereby obtaining pressure data corresponding to the plurality of firmness levels and the plurality of height levels. Then, a recommended firmness and a recommended height may be accurately obtained based on the pressure data corresponding to the plurality of firmness levels and the plurality of height levels in subsequent steps.

In one of the embodiments, the adjustable mattress includes a plurality of zones, with each zone corresponding to a different part of a human body. Moreover, each zone is provided with an airbag. Performing the plurality of firmness adjustments on the firmness of the adjustable mattress to obtain the pressure data collected by the pressure sensor array after each firmness adjustment includes: during a current firmness adjustment in the plurality of firmness adjustments, determining a target mattress firmness to be adjusted to during the current firmness adjustment; and adjusting the airbag in each zone of the adjustable mattress to the target mattress firmness, and obtaining first pressure data collected by the pressure sensor array under the target mattress firmness. Performing the plurality of head height adjustments on the adjustable mattress to obtain the pressure data collected by the pressure sensor array after each head height adjustment includes: during a current head height adjustment in the plurality of head height adjustments, determining a target head height to be adjusted to during the current head height adjustment; and adjusting an airbag in a zone of the adjustable mattress corresponding to the head to the target head height, and obtaining second pressure data collected by the pressure sensor array under the target head height.

Specifically, during the current firmness adjustment in the plurality of firmness adjustments, the target mattress firmness to be adjusted to during the current firmness adjustment is determined. For example, if the current firmness adjustment is a first firmness adjustment, it is determined that the firmness of the adjustable mattress needs to be adjusted to a preset 1-level firmness, and the this 1-level firmness in this case is the target mattress firmness; if the current firmness adjustment is a second firmness adjustment, it is determined that the firmness of the adjustable mattress needs to be adjusted to a 2-level firmness, and the 2-level firmness in this case is the target mattress firmness, and so on.

Further, after determining the target mattress firmness, the airbag in each zone of the adjustable mattress may be adjusted to this target mattress firmness and maintained for a period of time, thereby enabling the pressure sensor array to collect the pressure data under the target mattress firmness. For the convenience of description, the pressure data collected during the mattress firmness adjustment is referred to as "first pressure data" hereinafter.

After the plurality of firmness adjustments are performed, the plurality of head height adjustments may be performed. During the current head height adjustment in the plurality of head height adjustments, the target head height to be adjusted to during the current head height adjustment is determined. For example, if the current head height adjustment is a first head height adjustment, it is determined that the head height needs to be adjusted to a preset 1-level height, and the 1-level height in this case is the target head height; if the current head height adjustment is a second head height adjustment, it is determined that the head height needs to be adjusted to 2-level height, and the 2-level height in this case is the target head height, and so on. Further, after determining the target head height, the airbag in the zone of the adjustable mattress corresponding to the head may be adjusted to the target head height and maintained for a period of time, thereby enabling the pressure sensor array to collect the pressure data under the target head height. For the convenience of description, the pressure data collected during the head height adjustment process is referred to as "second pressure data" hereinafter.

After completing the pressure detection of the current round, the first pressure data to which each mattress firmness level corresponds and the second pressure data to which each head height level corresponds may be obtained.

In this embodiment, by adjusting the mattress firmness and head height in the step-by-step manner, the corresponding first pressure data may be collected for each level of mattress firmness, and the corresponding second pressure data may be collected for each level of head height. In this way, the obtained pressure data is more comprehensive.

In one of the embodiments, the sleep product state parameters include the mattress firmness and the pillow height. Determining the recommended sleep product state parameters according to the pressure data collected in the plurality of rounds of pressure detection and the physiological health data includes: obtaining the user's state parameter preferences, and determining a recommended first mattress firmness and a recommended first pillow height according to the state parameter preferences; determining a recommended second mattress firmness and a recommended second pillow height according to the pressure data; determining a recommended third mattress firmness and a recommended third pillow height according to the physiological health data; and determining a final recommended mattress firmness and a final recommended pillow height according to the recommended first mattress firmness, the recommended second mattress firmness, the recommended third mattress firmness, the recommended first pillow height, the recommended second pillow height, and the recommended third pillow height.

Specifically, after the user enters the lying cabin, the user may be guided to input preferred state data to obtain the state parameter preferences. For example, the user is guided to input the preferred mattress firmness and the preferred pillow height. The recommended mattress firmness and pillow height may be determined based on the state parameter preferences. For the convenience of description, the recommended mattress firmness and pillow height determined based on the state parameter preferences are respectively referred to as the recommended first mattress firmness and the recommended first pillow height below. For example, the preferred mattress firmness may be directly used as the recommended first mattress firmness, and the preferred pillow height may be directly used as the recommended first pillow height.

Further, after obtaining the first pressure data and the second pressure data, the recommended mattress firmness and pillow height may be obtained based on the first pressure data and the second pressure data. For the convenience of description, the recommended mattress firmness and pillow height determined based on the first pressure data and the second pressure data are respectively referred to as the recommended second mattress firmness and the recommended second pillow height below. For example, the first pressure data and the second pressure data may be input into a pre-trained state parameter recommendation model, which may then output the recommended second mattress firmness and the recommended second pillow height.

Further, after obtaining the physiological health data, the recommended mattress firmness and pillow height may be determined based on the physiological health data. For the convenience of description, the recommended mattress firmness and pillow height determined based on the physiological health data are respectively referred to as the recommended third mattress firmness and the recommended third pillow height below. For example, recommended mattress firmness levels and pillow heights corresponding to different heart rates and respiratory rates may be preset. After obtaining the user's heart rate and respiratory rate, a recommended third mattress firmness and a recommended third pillow height may be obtained based on the preset correspondence.

After obtaining the recommended first mattress firmness, the recommended second mattress firmness, the recommended third mattress firmness, the recommended first pillow height, the recommended second pillow height, and the recommended third pillow height, the final recommended mattress firmness and the final recommended pillow height may be determined based on the recommended first mattress firmness, the recommended second mattress firmness, the recommended third mattress firmness, the recommended first pillow height, the recommended second pillow height, and the recommended third pillow height.

For example, the final recommended mattress firmness may be obtained by calculating an average of the recommended first mattress firmness, the recommended second mattress firmness, and the recommended third mattress firmness. The final recommended pillow height is obtained by calculating an average of the recommended first pillow height, the recommended second pillow height, and the recommended third pillow height.

In one of the embodiments, after obtaining the final recommended mattress firmness and the final recommended pillow height, the final recommended pillow height may be further corrected based on the final recommended mattress firmness to obtain a corrected recommended mattress firmness that is matched with the final recommended mattress firmness. For example, a matching table may be obtained, which records a pillow height with which each mattress firmness is matched. Based on the matching table, the pillow height that is matched with the final recommended mattress firmness may be found. If a difference between the found pillow height and the final recommended pillow height is less than a preset difference threshold, no correction is performed. If the difference is greater than or equal to the preset difference threshold, the final recommended pillow height is corrected, so that a difference between the corrected recommended pillow height and the found pillow height is less than the preset difference threshold, and the final recommended mattress firmness and the corrected recommended pillow height are used as the recommended sleep product state parameters.

By correcting the final recommended pillow height, the recommended mattress firmness for the user may be matched with the recommended pillow height, thereby achieving a coordinated adjustment of pillow height and spinal support, and thus improving the user's sleep quality based on the coordinated adjustment.

In one of the embodiments, the user may also input preferred sleep postures, so that the first pressure data and the second pressure data are determined and obtained by synthesizing the preferred sleep postures and the preferred state data.

In this embodiment, the final recommended mattress firmness is obtained by synthesizing the recommended first mattress firmness, the recommended second mattress firmness, and the recommended third mattress firmness, which may make the final recommended mattress firmness more accurate. The final recommended pillow height is determined by synthesizing the recommended first pillow height, the recommended second pillow height, and the recommended third pillow height, which may make the final recommended pillow height more accurate.

In one of the embodiments, each round of pressure detection includes a plurality of mattress firmness adjustments and a plurality of head height adjustments. The pressure data collected in each round of pressure detection includes first pressure data collected in each of the plurality of mattress firmness adjustments and second pressure data collected in each of the plurality of head height adjustments. Determining the recommended second mattress firmness and the recommended second pillow height according to the pressure data includes: for each round of pressure detection in the plurality of rounds of pressure detection and for each mattress firmness adjustment and each head height adjustment in each round of pressure detection, processing first pressure data collected in a current mattress firmness adjustment to obtain processed pressure data corresponding to the current mattress firmness adjustment, where the processed pressure data includes at least one of average pressure data, maximum pressure data, and pressure distribution ratio data; processing second pressure data collected in a current head height adjustment to obtain processed pressure data corresponding to the current head height adjustment; determining a recommended fourth mattress firmness and a recommended fourth pillow height corresponding to a current round of pressure detection according to processed pressure data to which each mattress firmness adjustment corresponds and processed pressure data to which each head height adjustment corresponds in the current round of pressure detection; and determining the recommended second mattress firmness and the recommended second pillow height according to a recommended fourth mattress firmness and a recommended fourth pillow height to which each round of pressure detection corresponds.

Specifically, the following processing is performed for each round of pressure detection in the plurality of rounds of pressure detection. A current round of pressure detection is used as an example for illustration. In the current round of pressure detection, after obtaining the first pressure data, the first pressure data may be processed to obtain the processed pressure data. For example, when the user lies on their back on the adjustable mattress, after performing the plurality of adjustments on the mattress firmness to obtain the collected first pressure data after each adjustment, each set of first pressure data may be processed separately to obtain a plurality of sets of processed pressure data. The processed pressure data includes at least one of average pressure data, maximum pressure data, and pressure distribution ratio data. For example, an average pressure may be obtained by averaging the first pressure data, a maximum pressure may be obtained by extracting a maximum pressure value from the first pressure data. Moreover, since the adjustable mattress is provided with the plurality of zones, a pressure distribution ratio of each zone may be determined. A pressure distribution ratio of a current zone refers to a ratio of a pressure in the current zone to a sum of pressures in the zones.

Further, after obtaining the second pressure data, the second pressure data may be processed to obtain processed pressure data. For example, when the user lies on their back on the adjustable mattress, after performing the plurality of adjustments on the head height to obtain the collected second pressure data after each adjustment, each set of second pressure data may be processed separately to obtain a plurality of sets of processed pressure data.

Further, after obtaining processed pressure data to which each of the plurality of mattress firmness adjustments corresponds and processed pressure data to which each of the plurality of head height adjustments corresponds, the recommended fourth mattress firmness and the recommended fourth pillow height corresponding to the current round of pressure detection may be determined based on each set of processed pressure data. For example, optimal processed pressure data may be obtained, which may include an optimal average pressure, an optimal maximum pressure, and an optimal pressure distribution ratio. Processed pressure data that is closest to the optimal processed pressure data is screened out from the processed pressure data to which each of the plurality of mattress firmness adjustments corresponds, and is taken as first target processed pressure data. A mattress firmness corresponding to the first target processed pressure data is taken as the recommended fourth mattress firmness. That is, a mattress firmness to be adjusted to during a mattress firmness adjustment corresponding to the first target processed pressure data is taken as the recommended fourth mattress firmness corresponding to the current round of pressure detection. Correspondingly, processed pressure data that is closest to the optimal processed pressure data may be screened out from the processed pressure data to which each of the plurality of head height adjustments corresponds, and may be taken as second target processed pressure data. A head height corresponding to the second target processed pressure data is taken as the recommended fourth pillow height. That is, a head height to be adjusted to during a head height adjustment corresponding to the second target processed pressure data is taken as the recommended fourth pillow height corresponding to the current round of pressure detection.

Further, after obtaining the recommended fourth mattress firmness and the recommended fourth pillow height to which each round of pressure detection corresponds, the recommended second mattress firmness may be determined based on the recommended fourth mattress firmness to which each round of pressure detection corresponds. For example, an average value of the recommended fourth mattress firmness levels may be taken as the recommended second mattress firmness. Moreover, the recommended second pillow height may be determined based on the recommended fourth pillow height to which each round of pressure detection corresponds. For example, an average value of the recommended fourth pillow heights may be taken as the recommended second pillow height.

In one of the embodiments, the optimal processed pressure data may be obtained based on ergonomics. When a human body lies on a mattress, the pressure distribution of various body parts on the mattress has a certain ideal range. Generally, a hip pressure of approximately 30-40 mmHg is considered appropriate, while a shoulder pressure and a head pressure are relatively lower, usually between 20-30 mmHg. Therefore, the optimal average pressure, the optimal maximum pressure, and the optimal pressure distribution ratio may be obtained based on ergonomics, thereby obtaining the optimal processed pressure data.

In the present disclosure, since different rounds correspond to different sleep detection postures, the recommended second mattress firmness and the recommended second pillow height are obtained by synthesizing the recommended fourth mattress firmness and the recommended fourth pillow height to which each round of pressure detection corresponds, so that the recommended second mattress firmness and the recommended second pillow height are adaptable to different sleep postures.

In one of the embodiments, the lying cabin includes a display screen. The above method further includes: generating sleep posture pressure distribution maps based on the pressure data collected in the plurality of rounds of pressure detection; and displaying the sleep posture pressure distribution maps and the recommended sleep product state parameters on the display screen.

Specifically, the lying cabin includes the display screen. Since the pressure sensor array is a tiled array structure on the adjustable mattress, e.g., the pressure sensor array may be a 25×64 array structure, the pressure data collected by the pressure sensor array may include full-body pressure data of the user. Thus, a pressure distribution map may be obtained based on the pressure data, and this pressure distribution map is the sleep posture pressure distribution map. For example, when the user lies on their back, whenever a set of first pressure data is obtained, a pressure distribution map corresponding to a supine posture may be generated based on this set of first pressure data. Whenever a set of second pressure data is obtained, a pressure distribution map corresponding to a supine posture may also be generated based on this set of second pressure data. After the sleep posture pressure distribution map is obtained, the sleep posture pressure distribution map may be displayed on the display screen. For example, identifiers of all sleep posture pressure distribution maps may be displayed on the display screen, allowing the user to select which sleep posture pressure distribution map to display. Alternatively, some of the sleep posture pressure distribution maps may be directly displayed on the display screen.

Further, after the recommended sleep product state parameters are obtained, the recommended sleep product state parameters may also be displayed on the display screen.

In one of the embodiments, the user may further input their feelings about the current detection, and then the display screen may show the user's input feelings.

In this embodiment, by displaying the sleep posture pressure distribution maps, the user may intuitively perceive the impact of different mattress firmness levels and different pillow heights on pressure under different sleep postures. By displaying the recommended sleep product state parameters, the users may directly identify the recommended mattress firmness and pillow height to them.

In one of the embodiments, a system for recommending sleep product state parameters based on lying test is provided. The system includes a lying cabin, an electric bed frame, an adjustable mattress, and a display screen. The electric bed frame and the display screen are arranged in the lying cabin, and the adjustable mattress is placed on the electric bed frame. The adjustable mattress is provided with a pressure sensor array and a heart rate and respiration belt, and includes a plurality of zones. Each zone corresponds to a different part of a human body, and each zone is provided with an airbag. The electric bed frame is configured to independently lift a head and legs and adjust a tilt angle under the drive of servo motors for the head and legs. The adjustable mattress is configured to perform at least one of a firmness adjustment and a height adjustment based on the airbags in the zones during a plurality of rounds of pressure detection on a user. The pressure sensor array is configured to collect pressure data during each round of pressure detection. The heart rate and respiration belt is configured to collect the user's physiological health data. The display screen is configured to display sleep posture pressure distribution maps generated based on the pressure data and recommended sleep product state parameters generated based on the pressure data and the physiological health data.

In one of the embodiments, referring to FIG. 3, FIG. 3 is a schematic diagram of a system for recommending sleep product state parameters based on lying test in an embodiment.

An intelligent simulation cabin (i.e., lying cabin) has a closed design and integrates an annular sound-insulating panel, an openable and closable electric curtain, a surrounding LED guide light, and a three-dimensional voice prompt function.

The electric bed frame has the head and the legs that are respectively driven by the servo motors, a torque of ≥20 Nm, an adjustment speed of 5 mm/s, and an operating noise of <45 dB(A). The electric bed frame supports independent lifting of the head and the legs (with ±5 cm fine adjustment) and tilt angle adjustment.

For a flexible pressure sensor array (i.e., pressure sensor array), the 25×64 pressure sensor array communicates with the STM32H7 MCU via the SPI bus, with a sampling rate of 100 Hz and a resolution of 12 bits; each frame of data is approximately 25×64×12 bits ≈ 2.4 kB, and a transmission delay is <10 ms. A microcontroller unit (MCU) aggregates the data and uploads it to a dedicated router via an RJ45 (Cat5e) gigabit ethernet, with an actual throughput of 940-980 Mbps.

A dynamically adjustable airbag mattress (i.e., adjustable mattress) is divided into 7 major zones corresponding to the head, shoulders, back, waist, hips, legs, and feet. Each zone is provided with an independently controlled airbag, for supporting dynamic adjustment of firmness (0-50 kPa) and local height (±5 cm), with a valve response time of <50 ms. Each airbag is equipped with a micro differential pressure sensor with a typical error of 2.5 % FS. During a first use, each airbag is zero-calibrated using a reference weight (500 g ± 0.5 g), and a self-test is performed every 100 h of operation or before startup to ensure pressure adjustment accuracy.

The heart rate and respiration belt has a flexible micro-nano sensor of 80 cm × 15 cm, with a sampling rate of 200 Hz, a measuring range of 100 kPa, and a response time of <2 ms.

The display screen supports wireless screen projection and high definition multimedia interface (HDMI) direct connection, and is used to display a test process and a pressure cloud map comparison interface. The pressure cloud map comparison interface may include a plurality of sleep posture pressure distribution maps.

For a tablet and a dedicated router, the tablet communicates with the mattress via Bluetooth and exchanges data with a sensor control box within a local area network through a wireless network.

For a user terminal, the user scans a code to follow an official account and obtain a report and recommendation results, which include the sleep posture pressure distribution maps, the recommended state parameters, and personalized suggestions. The users may provide feedback on their experience.

The system further includes a cloud server and a third-party IoT platform. The cloud server is used to store the user's basic information (including preference information) and the user's experience information (including a recommendation report), and the cloud server may be used to manage the lying cabin. The third-party IoT platform is used to receive on and off commands issued by the cloud server to control the opening and closing of the lying cabin.

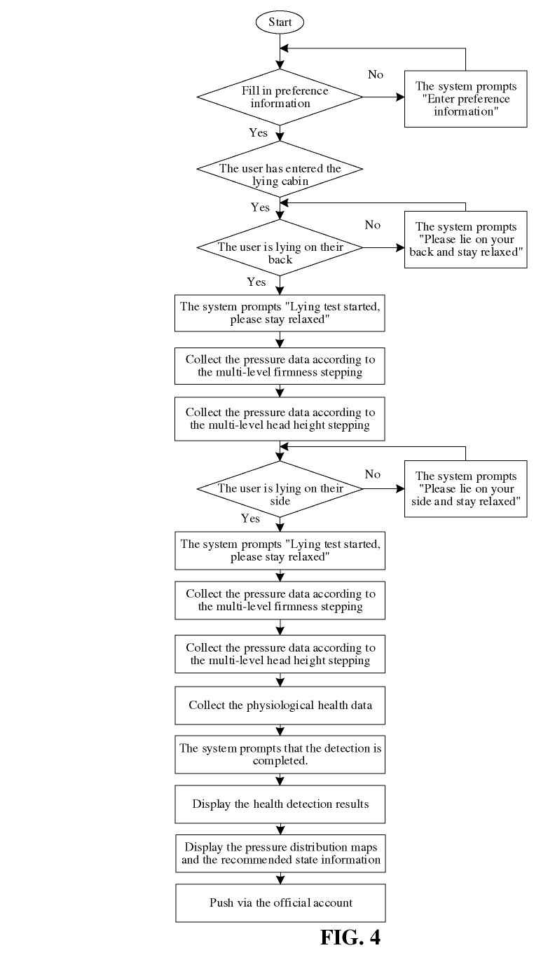

In one of the embodiments, referring to FIG. 4, FIG. 4 is an overall schematic flow diagram of a method for recommending sleep product state parameters based on lying test in an embodiment. Initially, in an initialization process, the adjustable mattress is restored to a flat state, and the electric bed frame is restored to a flat state. It is determined whether the user has filled in preference information, where the preference information includes the user's sleep posture preference and state parameter preference. If not filled in, information collection is conducted. The system prompts "Enter preference information", and the user then scans a code to input information such as sleep posture preference and mattress firmness preference. It is determined whether the user has entered the lying cabin; if yes, a cabin curtain is automatically closed, with a motor closing duration of <5 s and a cumulative noise of <40 dB(A). It is determined whether the user is lying on their back; if not, the system prompts "Please lie on your back and stay relaxed" and displays an example on a large screen to guide the user to lie on their back. If the user is lying on their back, the system prompts "Lying test started, please keep your body relaxed". According to the 7-level firmness stepping of the mattress, the firmness is simulated in a positive order from hard to soft, with each level lasting approximately 2.5 s. The first pressure data under each mattress firmness level is obtained. According to the 7-level height stepping of the head airbag (1 cm per level, with a total range of 0-6cm). The second pressure data under each head height level is obtained. A sleep posture pressure distribution map is generated based on the first pressure data or the second pressure data and displayed, and a refresh rate of the sleep posture pressure distribution map is 2 Hz, and an image rendering duration is <100 ms. The total test time for the supine posture is 20 s ± 1 s. It is determined whether the user is lying on their side; if not, the system prompts "Please lie on your side and stay relaxed" and displays an example on a large screen to guide the user to lie on their side. If the user is lying on their side, the system prompts "Lying test started, please keep your body relaxed". According to the 7-level firmness stepping of the mattress, the first pressure data is collected, and according to the 7-level height stepping of the head airbag, the second pressure data is collected. However, the firmness is simulated in a reverse order from soft to hard to verify data consistency. After the side-lying detection is completed, the system prompts "Next, physiological health data will be collected, which is expected to take about 4 minutes. Please return to the supine posture and stay relaxed". The user lies still for 4 minutes, and the heart rate and respiration belt collects the physiological health data. After the detection is completed, the head and the legs of the electric bed are lifted by 15° respectively, and the cabin curtain is automatically opened. The report is displayed, which includes the user's health detection results, sleep posture pressure distribution maps comparing 7 firmness levels, and the recommended state parameters. The results are synchronously pushed, and a complete report is sent to the user's mobile phone via the official account.

Currently, technologies in the field of mattress recommendation generally have the following problems.

Insufficient posture adaptability: existing technologies mostly rely on static human body characteristics (e.g., height, weight) and cannot dynamically adapt to the support needs of frequent body position changes during real sleep, leading to a large deviation between recommended results and actual usage scenarios. However, the lying test scheme based on the lying cabin in the present disclosure takes various sleeping postures into account and conducts detection under each sleep detection posture, so that the final recommended sleep product state parameters are obtained by synthesizing all sleep postures, thereby enabling the recommended sleep product state parameters to adapt to the support needs of frequent body position changes during real sleep.

Lack of head-pillow linkage: existing systems only provide suggestions on mattress firmness or model, ignoring the coordinated adjustment between pillow height and spinal support. Such defects result in poor user experience and failure to achieve the expected recommendation effect. However, the lying test scheme based on the lying cabin in the present disclosure not only recommends mattress firmness to users but also recommends a pillow height that is matched with the recommended mattress firmness to users. Thus, the pillow height may be adjusted in coordination with spinal support for the user, thereby further improving the user’s sleep quality.

Inability to evaluate recommendation effect: the design of existing systems only stays at the stage of outputting recommendation results and does not quantitatively demonstrate the improvement effect of the recommended scheme in practical application. The present disclosure may display the images generated during detection and the detection results by setting up the display screen.

It should be understood that although the steps in the flowcharts involved in the above embodiments are displayed in sequence according to the indication of arrows, these steps are not necessarily executed in the sequence indicated by the arrows. Unless explicitly stated herein, there is no strict order restriction on the execution of these steps, and these steps may be executed in other orders. Moreover, at least some of the steps in the flowcharts involved in the above embodiments may include a plurality of steps or stages. These steps or stages are not necessarily completed at the same time but may be executed at different times. The execution order of these steps or stages is not necessarily sequential, but may be executed alternately or in rotation with other steps or at least some of steps or stages in other steps.

Based on the same inventive concept, the embodiments of the present disclosure further provide a device for recommending sleep product state parameters based on lying test, for implementing the above method for recommending sleep product state parameters based on lying test. The implementation scheme for solving problems provided by the device is similar to the implementation scheme described in the above method. Therefore, the specific limitations in one or more embodiments of the device for recommending sleep product state parameters based on lying test provided below may refer to the limitations on the above method for recommending sleep product state parameters based on lying test, and will not be repeated here.

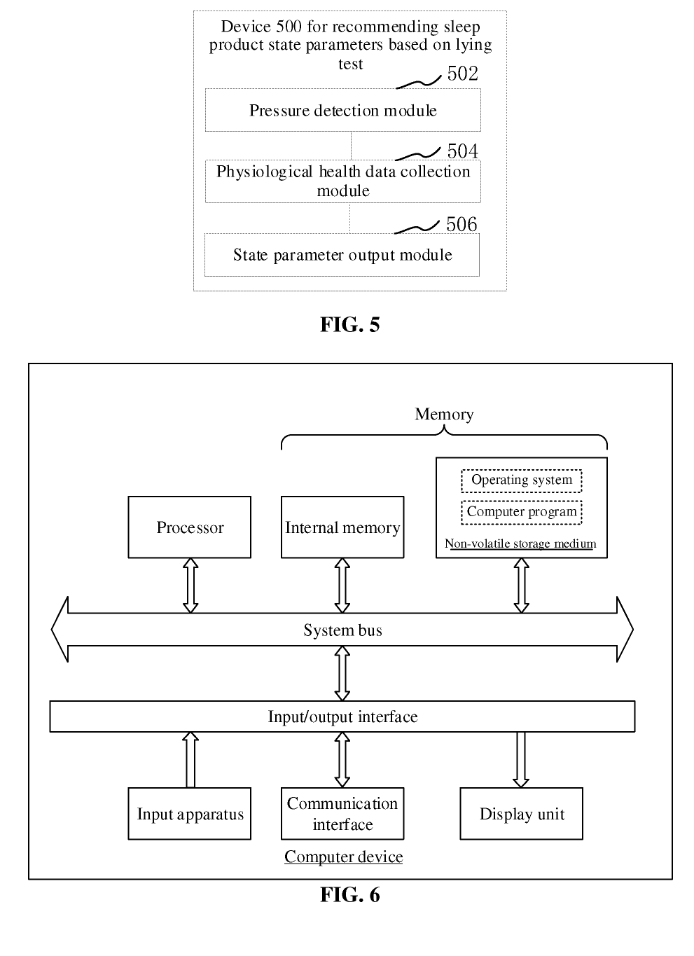

In one embodiment, as shown in FIG. 5, a device for recommending sleep product state parameters based on lying test is provided. The device includes a pressure detection module 502, a physiological health data collection module 504, and a state parameter output module 506.

The pressure detection module 502 is configured to: when the user enters the lying cabin, perform the plurality of rounds of pressure detection on the user, and in the current round of the plurality of rounds of pressure detection, determine the sleep detection posture corresponding to the current round; and guide the user to lie on the adjustable mattress in the sleep detection posture, and perform the step-by-step adjustment on the state parameters of the adjustable mattress to obtain the pressure data collected by the pressure sensor array.

The physiological health data collection module 504 is configured to collect the user’s physiological health data based on the heart rate and respiration belt.

The state parameter output module 506 is configured to determine the recommended sleep product state parameters according to the pressure data collected in the plurality of rounds of pressure detection and the physiological health data.

In one of the embodiments, the pressure detection module 502 is further configured to perform the plurality of adjustments on the firmness of the adjustable mattress to obtain the pressure data collected by the pressure sensor array after each firmness adjustment; and perform the plurality of head height adjustments on the adjustable mattress to obtain the pressure data collected by the pressure sensor array after each head height adjustment.

In one of the embodiments, the adjustable mattress includes the plurality of zones, with each zone corresponding to a different part of a human body. Moreover, each zone is provided with the airbag. The pressure detection module 502 is further configured to: during the current firmness adjustment in the plurality of firmness adjustments, determine the target mattress firmness to be adjusted to during the current firmness adjustment; adjust the airbag in each zone of the adjustable mattress to the target mattress firmness, and obtain the first pressure data collected by the pressure sensor array under the target mattress firmness; during the current head height adjustment in the plurality of head height adjustments, determine the target head height to be adjusted to during the current head height adjustment; and adjust the airbag in the zone of the adjustable mattress corresponding to the head to the target head height, and obtain the second pressure data collected by the pressure sensor array under the target head height.

In one of the embodiments, the sleep product state parameters include the mattress firmness and the pillow height. The state parameter output module 506 is further configured to: obtain the user’s state parameter preferences, and determine the recommended first mattress firmness and the recommended first pillow height according to the state parameter preferences; determine the recommended second mattress firmness and the second pillow height according to the pressure data; determine the recommended third mattress firmness and the recommended third pillow height according to the physiological health data; determine the final recommended mattress firmness and the final recommended pillow height according to the recommended first mattress firmness, the recommended second mattress firmness, the recommended third mattress firmness, the recommended first pillow height, the recommended second pillow height, and the recommended third pillow height.

In one of the embodiments, each round of pressure detection includes the plurality of mattress firmness adjustments and the plurality of head height adjustments. The pressure data collected in each round of pressure detection includes the first pressure data collected in each of the plurality of mattress firmness adjustments and the second pressure data collected in each of the plurality of head height adjustments. The state parameter output module 506 is further configured to: for each round of pressure detection in the plurality of rounds of pressure detection and for each mattress firmness adjustment and each head height adjustment in each round of pressure detection, process the first pressure data collected in the current mattress firmness adjustment to obtain the processed pressure data corresponding to the current mattress firmness adjustment, where the processed pressure data includes at least one of average pressure data, maximum pressure data, and pressure distribution ratio data; process the second pressure data collected in the current head height adjustment to obtain the processed pressure data corresponding to the current head height adjustment; determine the recommended fourth mattress firmness and the recommended fourth pillow height corresponding to the current round of pressure detection according to the processed pressure data to which each mattress firmness adjustment corresponds and the processed pressure data to which each head height adjustment corresponds in the current round of pressure detection; and determine the recommended second mattress firmness and the recommended second pillow height according to the recommended fourth mattress firmness and the recommended fourth pillow height to which each round of pressure detection corresponds.

In one of the embodiments, the state parameter output module 506 is further configured to: generate the sleep posture pressure distribution map based on the pressure data collected in the plurality of rounds of pressure detection; and display the sleep posture pressure distribution map and the recommended sleep product state parameters on the display screen.

All or part of the modules in the above device for recommending sleep product state parameters based on lying test may be implemented by software, hardware, or a combination thereof. The above modules may be embedded in or independent of a processor of a computer device in a form of hardware, or stored in a memory of the computer device in a form of software, so that the processor calls and execute operations corresponding to the above modules.

In one embodiment, a computer device is provided. The computer device may be a lying cabin. The computer device includes a processor, a memory, an input/output (I/O) interface, and a communication interface. The processor, the memory, and the input/output interface are connected through a system bus, and the communication interface is connected to the system bus through the input/output interface. The processor of the computer device is configured to provide computing and control capabilities. The memory of the computer device includes a non-volatile storage medium and an internal memory. The non-volatile storage medium stores an operating system, a computer program, and a database. The internal memory provides an environment for the operation of the operating system and the computer program in the non-volatile storage medium. The database of the computer device is configured to store the recommendation data of the sleep product state parameters based on lying test. The input/output interface of the computer device is configured to exchange information between the processor and an external device. The communication interface of the computer device is configured to communicate with an external terminal through a network connection. When the computer program is executed by the processor, the method for recommending sleep product state parameters based on lying test is implemented.

In one embodiment, a computer device is provided. The computer device may be a lying cabin, and its internal structure diagram may be as shown in FIG. 6. The computer device includes a processor, a memory, an input/output interface, a communication interface, a display unit, and an input apparatus. The processor, the memory, and the input/output interface are connected through a system bus, and the communication interface, the display unit, and the input apparatus are connected to the system bus through the input/output interface. The processor of the computer device is configured to provide computing and control capabilities. The memory of the computer device includes a non-volatile storage medium and an internal memory. The non-volatile storage medium stores an operating system and a computer program. The internal memory provides an environment for the operation of the operating system and the computer program in the non-volatile storage medium. The input/output interface of the computer device is configured to exchange information between the processor and an external device. The communication interface of the computer device is configured to communicate with an external terminal in a wired or wireless manner. The wireless manner may be implemented through WIFI, mobile cellular network, near field communication (NFC), or other technologies. When the computer program is executed by the processor, the l method for recommending sleep product state parameters based on lying test is implemented. The display unit of the computer device is configured to form a visually visible image, and may be a display screen, a projection device, or a virtual reality imaging device. The display screen may be a liquid crystal display or an electronic ink display. The input apparatus of the computer device may be a touch layer covered on the display screen, or keys, a trackball, or a touchpad provided on a housing of the computer device, or an external keyboard, touchpad, mouse, etc.

Those skilled in the art may understand that the structure shown in FIG. 6 is only a block diagram of a part of the structure related to the solution of the present disclosure, and does not constitute a limitation on the computer device to which the solution of the present disclosure is applied. A specific computer device may include more or fewer components than those shown in the figure, or combine some components, or have a different arrangement of components.

In one embodiment, a computer device is further provided. The computer device includes a memory and a processor. The memory stores a computer program, and when the processor executes the computer program, the steps of the above method embodiments are implemented.

In one embodiment, a computer-readable storage medium is provided. The computer-readable storage medium stores a computer program, and when the computer program is executed by a processor, the steps of the above method embodiments are implemented.

In one embodiment, a computer program product or a computer program is provided. The computer program product or the computer program includes computer instructions, and the computer instructions are stored in a computer-readable storage medium. A processor of a computer device reads the computer instructions from the computer-readable storage medium, and the processor executes the computer instructions, so that the computer device executes the steps of the above method embodiments.

It should be noted that the user information (including but not limited to user device information, user personal information, etc.) and data (including but not limited to data used for analysis, stored data, displayed data, etc.) involved in the present disclosure are all information and data authorized by the user or fully authorized by all parties, and the collection, use, and processing of relevant data must comply with the relevant laws, regulations, and standards of the corresponding countries and regions.

A person of ordinary skill in the art may understand that all or part of the processes in the methods of the above embodiments may be completed by instructing relevant hardware through a computer program. The computer program may be stored in a non-volatile computer-readable storage medium. When the computer program is executed, it may include the processes of the above method embodiments. Any reference to a memory, database, or other medium used in the embodiments provided in the present disclosure may include at least one of non-volatile memory and volatile memory. The non-volatile memory may include read-only memory (ROM), magnetic tape, floppy disks, flash memory, optical memory, high-density embedded non-volatile memory, resistive random access memory (ReRAM), magnetoresistive random access memory (MRAM), ferroelectric random access memory (FRAM), phase change memory (PCM), graphene memory, etc. The volatile memory may include random access memory (RAM) or external cache memory, etc. For illustration rather than limitation, RAM may be in various forms, such as static random access memory (SRAM) or dynamic random access memory (DRAM). The database involved in the embodiments provided in the present disclosure may include at least one of relational database and non-relational database. The non-relational database may include blockchain-based distributed database, etc., which is not limited thereto. The processor involved in the embodiments provided in the present disclosure may be general-purpose processor, central processing unit, graphics processing unit, digital signal processor, programmable logic device, quantum computing-based data processing logic device, etc., which is not limited thereto.

The technical features of the above embodiments may be combined arbitrarily. To make the description concise, not all possible combinations of the technical features in the above embodiments are described; however, as long as there is no contradiction in the combination of these technical features, the combinations should be considered within the scope covered by this specification.

The above embodiments only represent several implementations of the present disclosure, and their descriptions are specific and detailed, but they should not be construed as limiting the patent scope of the present disclosure. It should be noted that for those of ordinary skill in the art, several modifications and improvements can be made without departing from the concept of the present disclosure, and are all fall within the protection scope of the present disclosure. Therefore, the protection scope of the present disclosure should be subject to the appended claims.

What is claimed is:

1. A method for recommending sleep product state parameters based on lying test, wherein the method is applied to a lying cabin, the lying cabin comprises an adjustable mattress, and a pressure sensor array and a heart rate and respiration belt are provided on the adjustable mattress; and the method comprises:
in response to a user entering the lying cabin, performing a plurality of rounds of pressure detection on the user, and in a current round of the plurality of rounds of pressure detection, determining a sleep detection posture corresponding to the current round;
guiding the user to lie on the adjustable mattress in the sleep detection posture, and performing a step-by-step adjustment on state parameters of the adjustable mattress to obtain pressure data collected by the pressure sensor array;
collecting physiological health data of the user based on the heart rate and respiration belt; and
determining recommended sleep product state parameters according to the pressure data collected in the plurality of rounds of pressure detection and the physiological health data.

2. The method according to claim 1, wherein performing the step-by-step adjustment on the state parameters of the adjustable mattress to obtain the pressure data collected by the pressure sensor array comprises:
performing a plurality of firmness adjustments on a firmness of the adjustable mattress to obtain pressure data collected by the pressure sensor array after each firmness adjustment; and
performing a plurality of head height adjustments on the adjustable mattress to obtain pressure data collected by the pressure sensor array after each head height adjustment.

3. The method according to claim 2, wherein the adjustable mattress comprises a plurality of zones, with each zone corresponding to a different part of a human body, and each zone is provided with an airbag; and performing the plurality of firmness adjustments on the firmness of the adjustable mattress to obtain the pressure data collected by the pressure sensor array after each firmness adjustment comprises:
during a current firmness adjustment in the plurality of firmness adjustments, determining a target mattress firmness to be adjusted to during the current firmness adjustment; and
adjusting the airbag in each zone of the adjustable mattress to the target mattress firmness, and obtaining first pressure data collected by the pressure sensor array under the target mattress firmness; and
performing the plurality of head height adjustments on the adjustable mattress to obtain the pressure data collected by the pressure sensor array after each head height adjustment comprises:
during a current head height adjustment in the plurality of head height adjustments, determining a target head height to be adjusted to during the current head height adjustment; and
adjusting an airbag in a zone of the adjustable mattress corresponding to a head to the target head height, and obtaining second pressure data collected by the pressure sensor array under the target head height.

4. The method according to claim 1, wherein the sleep product state parameters comprise a mattress firmness and a pillow height; and determining the recommended sleep product state parameters according to the pressure data collected in the plurality of rounds of pressure detection and the physiological health data comprises:
obtaining state parameter preferences of the user, and determining a recommended first mattress firmness and a recommended first pillow height according to the state parameter preferences;
determining a recommended second mattress firmness and a recommended second pillow height according to the pressure data;
determining a recommended third mattress firmness and a recommended third pillow height according to the physiological health data; and
determining a final recommended mattress firmness and a final recommended pillow height according to the recommended first mattress firmness, the recommended second mattress firmness, the recommended third mattress firmness, the recommended first pillow height, the recommended second pillow height, and the recommended third pillow height.

5. The method according to claim 4, wherein each round of pressure detection comprises a plurality of mattress firmness adjustments and a plurality of head height adjustments; pressure data collected in each round of pressure detection comprises first pressure data collected in each of the plurality of mattress firmness adjustments and second pressure data collected in each of the plurality of head height adjustments, wherein
determining the recommended second mattress firmness and the recommended second pillow height according to the pressure data comprises:
for each round of pressure detection in the plurality of rounds of pressure detection and for each mattress firmness adjustment and each head height adjustment in each round of pressure detection, processing first pressure data collected in a current mattress firmness adjustment to obtain processed pressure data corresponding to the current mattress firmness adjustment, where the processed pressure data comprises at least one of average pressure data, maximum pressure data, and pressure distribution ratio data;
processing second pressure data collected in a current head height adjustment to obtain processed pressure data corresponding to the current head height adjustment;
determining a recommended fourth mattress firmness and a recommended fourth pillow height corresponding to a current round of pressure detection according to processed pressure data to which each mattress firmness adjustment corresponds and processed pressure data to which each head height adjustment corresponds in the current round of pressure detection; and
determining the recommended second mattress firmness and the recommended second pillow height according to a recommended fourth mattress firmness and a recommended fourth pillow height to which each round of pressure detection corresponds.

6. The method according to claim 1, wherein the lying cabin comprises a display screen; and the method further comprises:
generating sleep posture pressure distribution maps based on the pressure data collected in the plurality of rounds of pressure detection; and
displaying the sleep posture pressure distribution maps and the recommended sleep product state parameters on the display screen.

7. A system for recommending sleep product state parameters based on lying test, wherein the system comprises a lying cabin, an electric bed frame, an adjustable mattress, and a display screen, wherein
the electric bed frame and the display screen are arranged in the lying cabin, and the adjustable mattress is placed on the electric bed frame; the adjustable mattress is provided with a pressure sensor array and a heart rate and respiration belt, and the adjustable mattress comprises a plurality of zones; each zone corresponds to a different part of a human body, and each zone is provided with an airbag;
the electric bed frame is configured to independently lift a head and legs and adjust a tilt angle under a drive of servo motors for the head and the legs;
the adjustable mattress is configured to perform at least one of a firmness adjustment and a height adjustment based on airbags arranged in the zones during a plurality of rounds of pressure detection on a user;
the pressure sensor array is configured to collect pressure data during each round of pressure detection;
the heart rate and respiration belt is configured to collect physiological health data of the user; and
the display screen is configured to display sleep posture pressure distribution maps generated based on the pressure data and recommended sleep product state parameters generated based on the pressure data and the physiological health data.

8. A computer device, comprising a memory and a processor, wherein the memory stores a computer program, wherein when the processor executes the computer program, the steps of the method according to claim 1 are implemented.

9. A computer-readable storage medium, storing a computer program, wherein when the computer program is executed by a processor, the steps of the method according to claim 1 are implemented.

10. A computer program product, comprising a computer program, wherein when the computer program is executed by a processor, the steps of the method according to claim 1 are implemented.
